(19) United States
(12) Patent Application Publication
(10) Pub. No.: US 2018/0264209 A1
(43) Pub. Date: Sep. 20, 2018

(54) NEBULIZERS HAVING YAWN DETECTION AND USES THEREOF

(71) Applicant: OMEGA LIFE SCIENCE LTD., Migdal Haemek (IL)

(72) Inventor: Miron Hazani, Haifa (IL)

(73) Assignee: OMEGA LIFE SCIENCE LTD., Migdal Haemek (IL)

(21) Appl. No.: 15/571,271

(22) PCT Filed: May 9, 2016
(86) PCT No.: PCT/IL2016/050491

§ 371 Date: Nov. 2, 2017

Related U.S. Application Data
(60) Provisional application No. 62/159,315, filed on May 10, 2015.
(60) Provisional application No. 62/271,366, filed on Dec. 28, 2015.

(51) Int. Cl. A61M 15/00

(52) U.S. Cl. CPC A61M 15/009; A61M 2205/33; A61B 5/1176; A61M 2205/3331; A61M 2205/3375; A61M 2230/63

(57) ABSTRACT

The present disclosure generally relates to the field of controllable nebulizers for aerosol generation. The nebulizer comprises an aerosol release mechanism configured to release aerosols based on control signals received from a controller based on yawn sensor measurements of the patient. The aerosol release during deep and full inhalation, as in yawns, improves drug delivery to the lungs and consequently may lead to reduction of drug dosage.

### TECHNICAL FIELD

The present disclosure generally relates to the field of nebulizers for aerosol generation and methods of using same for treating diseases and disorders.

### BACKGROUND

Nebulizers are commonly used for delivering aerosol medication to patients via the respiratory system. Two main goals of inhalation include promoting a more rapid onset of drug action and decreasing doses of medications. Currently there are three major categories of dispensers for lung deposition of drugs: pressurized metered-dose inhalers (PMDIs), dry powder inhalers (DPI) and nebulizers.

To limit drug waste during exhalation, breath-enhanced nebulizers, breath-actuated nebulizers (BANs), and nebulizers with an attached storage bag and a one-way mouthpiece valve have been developed. For example, the breath actuated AeroEclipse® II nebulizer, creates aerosol only during the inspiratory phase.

Conventional aerosol delivery systems and the availability of new technologies have led to the development of “intelligent” nebulizers, such as the I-neb Adaptive Aerosol Delivery (AAD) System. This system has been designed to continuously adapt to changes in the patient's breathing pattern, and to pulse aerosol only during the inspiratory part of the breathing cycle.

With regards to PMDIs, poor coordination of canister actuation and inspiration often prevents adequate metered-dose inhaler (MDI) usage by patients. Breath-actuated inhalers (BAIs) have been developed to prevent this problem. BAIs also deliver a pressurized aerosol metered dose of drug, and are automatically actuated when the user inhales through the mouthpiece.

A yawn is a reflex known to trigger a deep inhalation.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

In some embodiments there is provided a system for aerosols delivery, the system comprising an aerosol delivery device comprising a controllable aerosol release mechanism configured to release aerosols based on a control signal; a yawn detector configured to provide a yawn indicative signal in a subject; and a processing circuitry configured to identify a yawn based on said yawn indicative signal and to provide a control signal to said aerosol release mechanism, thereby affect release of aerosols from said device.

In some embodiments the yawn is facilitated by a facial recognition program capable of recognizing facial gestures associated with yawning.

In some embodiments the processing circuitry is further configured to stimulate yawning in the subject.

In some embodiments the system further comprises a yawn stimulator configured to stimulate the yawning.

In some embodiments the yawn stimulator is configured to provide a still image, dynamic image, sound, scent, flavor, sensation or any combination thereof.

Without wishing to be bound by any theory or mechanism, the yawn stimulating signals may induce yawning through a “contagious yawning” mechanism.

In some embodiments the yawning stimulation is activated by the subject or a caregiver.

In some embodiments the yawning stimulation is activated automatically.

In some embodiments the aerosol delivery device is an inhaler or a nebulizer.

In some embodiments the aerosol delivery device is selected from the group consisting of: a pressurized meter dose inhaler, dry particle inhaler or soft mist inhaler.

In some embodiments the facial gestures comprise a deep inhalation maneuver.

It is to be understood that a yawn includes a phase of deep inhalation. Typically, this phase occurs just before the widest opening of the mouth and closing of the eyes take place.

In some embodiments the processing circuitry is configured to predict a yawn based on the yawn indicative signal, and to provide a control signal to the aerosol release mechanism, based on said prediction, thereby schedule release of aerosols from the aerosol delivery device.

In some embodiments the aerosol release is a bolus aerosol release.

In some embodiments the aerosol comprises a pharmaceutical composition.

In some embodiments there is provided a use of a system as described herein in the treatment of a pulmonary disease or disorder.

In some embodiments the aerosols comprise a pharmaceutical composition for the treatment of said pulmonary disease or disorder.

In some embodiments there is provided a method of delivering aerosols to a subject in need thereof, the method comprises: providing an aerosol delivery device functionally associated with a processing circuitry having a yawn detector, wherein said aerosol delivery device comprises a controllable aerosol release mechanism; and actuating the controllable aerosol release mechanism, upon the processing circuitry receiving indication of a yawn from the yawn detector, thereby releasing aerosols from the aerosol delivery device.

In some embodiments the method further comprises stimulating a yawn in said subject.

In some embodiments receiving indication of a yawn comprises applying a facial recognition program capable of recognizing facial gestures associated with yawning.

In some embodiments stimulating a yawn comprises providing yawn stimulating signals.

In some embodiments the yawn stimulating signals are selected from the group consisting of still image, dynamic image, sound, scent, flavor, sensation or a combination thereof.

In some embodiments the method is for the treatment of a respiratory disease or disorder.

In some embodiments the disease or disorder is a pulmonary disease or disorder.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a functional block diagram of a system for aerosols delivery, according to some embodiments;

FIG. 2 schematically illustrates a system for aerosols delivery, according to some embodiments;

FIG. 3 schematically illustrates a system for aerosols delivery, according to some embodiments;

FIG. 4 schematically illustrates a device for aerosols delivery, according to some embodiments;

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

In some embodiments there are provided herein systems and methods for aerosols delivery. The system comprises an aerosol delivery device, such as, but not limited to a nebulizer or an inhaler; and a processing circuitry configured to identify a yawn and to trigger a release of aerosols from said device upon identification of a yawn in a subject.

The combination of an aerosol delivery device with a processing circuitry configured to identify a yawn allows optimal timing of an aerosol release to a subject in need thereof. In particular, this combination allows release of aerosol in the midst of a deep inhalation, which occurs during a yawn. Scheduling an aerosol delivery at the stage of deep inhalation improves the efficiency of delivering aerosol to the subject lungs.

In some embodiments there is provided a system for aerosols delivery, the system comprising an aerosol delivery device comprising a controllable aerosol release mechanism configured to release aerosols based on a control signal; a yawn detector configured to provide a yawn indicative signal in a subject; and a processing circuitry configured to identify a yawn based on said yawn indicative signal and to provide a control signal to said aerosol release mechanism, thereby affect release of aerosols from said device.

The terms “aerosols” and “aerosol” as used herein are interchangeable and describe a nebulized solution or suspension consisting of very fine particles carried by a gas, which typically consists of air. The suspensions may be prepared from of a formulation in an inert liquid, such as water, wherein the formed dispersion usually comprises wet microspheres in air. Generally, aerosols include a gas-borne suspended phase, which is capable of being inhaled into the bronchioles or nasal passages. Aerosols may be produced, for example, by a metered dose inhaler or nebulizer, by a mist sprayer, or specifically by an aerosol delivery device according to the present invention. Typically, medical aerosols include dry powder compositions of pharmaceutical agent(s), employed in respiratory therapy for the treatment of medical conditions. Conditions susceptible to treatment with aerosols include, but are not limited to, bronchospasms, loss of compliance, mucosal edema, pulmonary infections and the like.

The term “nebulize” is used as a synonym for “transforming a liquid into an aerosol”. Typically, nebulization occurs within a chamber where the aerosol is produced, by utilizing a source of energy, such as, a pneumatic or piezo-electric, which creates the aerosol.

In some embodiments, the aerosols is consisting of water. In some embodiments, the aerosols include a pharmaceutical composition. In some embodiments, the pharmaceutical composition is in a form of a dry powder.

A yawn is a reflex consisting of the simultaneous deep inhalation of air and the stretching of the eardrums, followed by an exhalation of breath. The average duration of a yawn is about six seconds, during which, the heart rate increases significantly.

Yawning most often occurs in adults immediately before and after sleep, during tedious activities and as a result of its contagious quality. It is commonly associated with tiredness, stress, sleepiness, or even boredom and hunger, though studies show it may be linked to the cooling of the brain. Yawns are often characterized by specific facial gestures, such as a wide opening of the mouth, stretching of the cheeks and eyebrows, opening or closing the eyelids, widening of the nostrils and wrinkling of the forehead. During yawning a person inhales deeply.

Due to the psychological effect of yawning, a yawn may be triggered by suggestive means. Yawning is often triggered by others yawning (e.g., seeing a person yawning, hearing the sound of yawning and even discussing yawning) and is a typical example of positive feedback. Moreover, it was found that yawning in rats is related to the sense of smell and can be triggered by exposing the animals to specific odors.

In some embodiments, the processing circuitry is configured to identify a yawn based on recognition of at least one facial gesture associated with yawning. In some embodiments, the identification of a yawn in a subject is based on recognition of at least two facial gestures associated with yawning. In some embodiments, the identification of a yawn in a subject is based on recognition of at least three facial gestures associated with yawning.

In some embodiments, the yawn indicative signal is provided based on recognition of at least one facial gesture associated with yawning. In some embodiments, the yawn indicative signal is provided based on recognition of at least two facial gestures associated with yawning.

In some embodiments, the identification of a yawn in a subject is based on recognition of at least one sound of the subject. In some embodiments, the yawn indicative signal is provided based recognition of at least one sound of the subject.

In some embodiments, the identification of a yawn in a subject is based on pneumatic pressure. In some embodiments, the yawn indicative signal is provided based on pneumatic pressure.

Identification of a pneumatic pressure as a signal corresponding to yawning and/or formation of a yawn includes, but is not limited to, the measurement of pressure at the mouth/mouth cavity. Typically, yawning is associated with a temporary decrease in pressure. Thus, identification of a reduced pressure or a negative change in pressure relates to the phenomenon of yawning.

In some embodiments, the identification of a yawn in a subject is based on recognition of a change in pneumatic pressure. In some embodiments, the yawn indicative signal is provided based on recognition of a change in pneumatic pressure.

In some embodiments, the change in pneumatic pressure comprises a decrease in pneumatic pressure.

In some embodiments, the identification of a yawn in a subject is based on recognition of a change in pneumatic flow. In some embodiments, the yawn indicative signal is provided based on recognition of a change in pneumatic flow.

In some embodiments, the change in pneumatic flow comprises an increase in pneumatic flow.

In some embodiments, the processing circuitry is configured to predict a yawn based on the yawn indicative signal, and to provide a control signal to the aerosol release mechanism, based on said prediction, thereby scheduling release of aerosols from the aerosol delivery device.

In some embodiments the prediction is based on recognition of at least one facial gesture associated with yawning. In some embodiments the prediction is based on recognition of at least two facial gestures associated with yawning.

In some embodiments the prediction is based on recognition of at least one sound of the subject.

In some embodiments the prediction is based on recognition a change in pneumatic pressure.

In some embodiments the prediction is based on recognition a change in pneumatic flow.

In some embodiments the yawn detector is configured to detect motion, sound, pneumatic flow, pneumatic pressure or any combination thereof. Each possibility represents a separate embodiment.

In some embodiments the yawn detector is configured to detect motion.

In some embodiments the yawn detector comprises a camera, a microphone, an air flow meter, a pressure gauge or any combination thereof.

In some embodiments the yawn detector comprises a camera.

In some embodiments the identification of the yawn is facilitated by a facial recognition program capable of recognizing facial gestures associated with yawning.

In some embodiments the facial recognition program is installed in the processing circuitry.

In some embodiments the processing circuitry comprises a mobile electronic device. In some embodiments the processing circuitry includes in a mobile electronic device.

In some embodiments the processing circuitry comprises a personal computer, a desktop computer, a laptop computer, a tablet, a phablet, smartwatch or a smartphone. Each possibility represents a separate embodiment. In some embodiments the processing circuitry comprises a tablet, a phablet a smartwatch or a smartphone.

In some embodiments the facial recognition program is a software or an application. In some embodiments the facial recognition program is embedded in the processing circuitry.

In some embodiments the facial recognition program includes a yawning detection algorithm.

In some embodiments the facial recognition program is configured to monitor the facial gestures.

In some embodiments the facial recognition program is configured to analyze data relating to the facial gestures associated with yawning.

In some embodiments the facial recognition program is further configured to decide if a yawn occurs. In some embodiments said facial recognition program is configured to decide when a yawn occurs. In some embodiments said facial recognition program is configured to predict when a yawn is expected to occur.

In some embodiments said facial recognition program is configured to decide if a yawn occurs based on at least one facial gesture associated with a yawn. In some embodiments said facial recognition program is configured to decide if a yawn occurs based on at least two facial gestures associated with a yawn.

In some embodiments said facial recognition program is configured to decide if a yawn occurs based on recognition of at least one sound of the subject.

In some embodiments said facial recognition program is configured to decide if a yawn occurs based on recognition a change in pneumatic pressure.

In some embodiments said facial recognition program is configured to decide if a yawn occurs based on recognition a change in pneumatic flow.

In some embodiments said facial recognition program is configured to decide when a yawn occurs based on at least one facial gesture associated with a yawn. In some embodiments said facial recognition program is configured to decide when a yawn occurs based on at least two facial gestures associated with a yawn.

In some embodiments said facial recognition program is configured to decide when a yawn occurs based on recognition of at least one sound of the subject.

In some embodiments said facial recognition program is configured to decide when a yawn occurs based on recognition a change in pneumatic pressure.

In some embodiments said facial recognition program is configured to decide when a yawn occurs based on recognition a change in pneumatic flow.

In some embodiments said facial recognition program is configured to predict when a yawn occurs based on at least one facial gesture associated with a yawn. In some embodiments said facial recognition program is configured to predict when a yawn occurs based on at least two facial gestures associated with a yawn.

In some embodiments said facial recognition program is configured to predict when a yawn occurs based on recognition of at least one sound of the subject.

In some embodiments said facial recognition program is configured to predict when a yawn occurs based on recognition a change in pneumatic pressure.

In some embodiments said facial recognition program is configured to predict when a yawn occurs based on recognition a change in pneumatic flow.

In some embodiments the facial gestures comprise a deep inhalation maneuver.

In some embodiments the facial gestures associated with a yawn comprise pre-yawning facial gestures.

In some embodiments the facial recognition program is further configured to provide a command to the processing circuitry to provide a control signal to the aerosol delivery device, thereby affect release of aerosols from the device.

In some embodiments the command is provided based on said decision obtained upon occurrence of a yawn. In some embodiments the command is provided based on said decision when a yawn occurs. In some embodiments the command is provided based on said prediction when a yawn occurs.

In some embodiments said command is given immediately upon the decision if or when a yawn occurs.

The term “immediately” as used herein refers to a time scale of fractions of a second or at most a few seconds, and no more than six seconds, which is the estimated duration of a yawn.

In some embodiments said command is given upon said prediction when a yawn occurs, such that the release of aerosols by the aerosol delivery device occurs during deep inhalation. In some embodiments said command is given upon said prediction when a yawn occurs, such that the release of aerosols by the aerosol delivery device occurs during the first second (namely, during 0 sec<tₐₑᵣₒₛₒₗ≤1 sec, wherein tₐₑᵣₒₛₒₗ refers to the time of aerosol release) of yawning. In some embodiments said command is given upon said prediction when a yawn occurs, such that the release of aerosols by the aerosol delivery device occurs after the first second of yawning and before or during the consecutive second (namely, during 1 sec<tₐₑᵣₒₛₒₗ≤2 sec). In some embodiments said command is given upon said prediction when a yawn occurs, such that the release of aerosols by the aerosol delivery device occurs during the third second of yawning (namely, during 2 sec<tₐₑᵣₒₛₒₗ≤3 sec). In some embodiments said command is given upon said prediction when a yawn occurs, such that the release of aerosols by the aerosol delivery device occurs during the fourth second of yawning (namely, during 3 sec<tₐₑᵣₒₛₒₗ≤4 sec). In some embodiments said command is given upon said prediction when a yawn occurs, such that the release of aerosols by the aerosol delivery device occurs during the fifth second of yawning (namely, during 4 sec<tₐₑᵣₒₛₒₗ≤5 sec). In some embodiments said command is given upon said prediction when a yawn occurs, such that the release of aerosols by the aerosol delivery device occurs during the sixth second of yawning (namely, during 5 sec<tₐₑᵣₒₛₒₗ≤6 sec).

In some embodiments said command is given upon said prediction when a yawn occurs, such that the release of aerosols by the aerosol delivery device occurs during the first two seconds of yawning (namely, during 0 sec<tₐₑᵣₒₛₒₗ≤2 sec); the first three seconds of yawning (namely, during 0 sec<tₐₑᵣₒₛₒₗ≤3 sec); the first four seconds of yawning (namely, during 0 sec<tₐₑᵣₒₛₒₗ≤4 sec); the first five seconds of yawning (namely, during 0 sec<tₐₑᵣₒₛₒₗ≤5 sec); or the first six seconds of yawning (namely, during 0 sec<tₐₑᵣₒₛₒₗ≤6 sec).

In some embodiments said facial gestures include, but are not limited to, layout, positions, movements, shift, shapes, alterations, adjustments, arrangements, orientations, locations, contractions, expansions, spreading, stretching, enlargement, distortion, deviation, maneuvers, outline and/or appearance of at least one element of the face of a user. Each possibility represents a separate embodiment.

In some embodiments said elements include, but not are limited to, mouth, lips eye(s), jaw, ear(s), nose, nostrils, cheeks, eyebrow(s), neck facial skin and/or forehead. Each possibility represents a separate embodiment.

In some embodiments the facial gestures associated with a yawn include any one or more of wide opening of the mouth, expansion of the nostrils and closing of the eyes.

In some embodiments the processing circuitry is functionally associated with a camera. In some embodiments the processing circuitry is connected to the camera by an electric cable. In some embodiments the processing circuitry is wirelessly associated with the camera. In some embodiments the camera is contained within the processing circuitry.

In some embodiments the facial gestures associated with a yawn are provided by the yawn detector. In some embodiments the facial gestures associated with a yawn are provided by the camera.

In some embodiments the processing circuitry comprises a non-transitory memory storage unit. In some embodiments the processing circuitry is configured to send and receive computer readable data to the non-transitory memory storage unit.

In some embodiments the computer readable data includes data specific to a user, in order to identify the subject and thus identify facial gestures thereof. In some embodiments the data specific to a user is derived from photos of the user. In some embodiments the photos of the user include photos of the user yawning. In some embodiments the photos of the user include photos of the user not yawning. In some embodiments the photos of the user include photos of the user yawning and photos of the user not yawning.

In some embodiments the photos of the user are provided by the yawn detector. In some embodiments the photos of the user are provided by the camera.

In some embodiments the data specific to a user is derived from sounds of the user. In some embodiments the sounds of the user include yawning sounds of the user.

In some embodiments the sounds of the user are provided by the yawn detector. In some embodiments the sounds of the user are provided by the microphone.

In some embodiments the processing circuitry is equipped to receive the data specific to a user. In some embodiments the data specific to a user is derived from photos of the user, provided by the detector. In some embodiments the data specific to a user is derived audible records of the user, provided by the detector.

In some embodiments the processing circuitry is equipped to receive data relating to the facial gestures at least once per second. In some embodiments the processing circuitry is equipped to receive data relating to the facial gestures at least twice per second. In some embodiments the processing circuitry is equipped to receive data relating to the facial gestures at least five times per second. In some embodiments the processing circuitry is equipped to receive data relating to the facial gestures at least 10 times per second. In some embodiments the processing circuitry is equipped to receive data relating to the facial gestures at least 25 times per second. In some embodiments the processing circuitry is equipped to receive data relating to the facial gestures at least 50 times per second. In some embodiments the processing circuitry is equipped to receive data relating to the facial gestures at least 100 times per second. In some embodiments the processing circuitry is equipped to receive data relating to the facial gestures at least 1,000 times per second.

In some embodiments the processing circuitry is further configured to stimulate yawning in the subject.

In some embodiments the system further comprises a yawn stimulator configured to stimulate yawning.

In some embodiments the processing circuitry is functionally associated with the yawn stimulator.

In some embodiments the processing circuitry is connected to the yawn stimulator by an electric cable. In some embodiments the processing circuitry is wirelessly associated with the yawn stimulator. In some embodiments the yawn stimulator is contained within the processing circuitry.

In some embodiments the yawn stimulator is configured to provide a still image, dynamic image, sound, scent, flavor, sensation, or any combination thereof. Each possibility represents a separate embodiment.

In some embodiments the yawn stimulator is configured to provide a still image, a dynamic image, sound or any combination thereof.

In some embodiments the yawn stimulator is configured to provide a still image.

In some embodiments the yawn stimulator is configured to provide a dynamic image.

In some embodiments the yawn stimulator is configured to provide sound(s).

In some embodiments the yawn stimulator is configured to provide a still image and sound(s).

In some embodiments the yawn stimulator is configured to provide a dynamic image and sound(s).

In some embodiments the yawn stimulator is configured to provide a change in temperature. In some embodiments the change in temperature comprises an increase in temperature.

In some embodiments the yawn stimulator comprises a display element.

In some embodiments the display element comprises a screen.

In some embodiments the yawn stimulator comprises an audio element.

In some embodiments the yawn stimulator comprises a display element and/or an audio element.

In some embodiments the audio element comprises at least one speaker.

In some embodiments the sound includes sounds of humans yawning, sounds of animals yawing, pronunciations and/or repetitions of words and/or sentences, monotonic sounds and/or stories and the like. Each possibility represents a separate embodiment.

In some embodiments the image includes a video, a figure or both. In some embodiments the image includes a video. In some embodiments the image includes a figure. In some embodiments the image includes a video and a figure. In some embodiments the figure includes a plurality of figures. In some embodiments the video includes a plurality of videos.

In some embodiments the video and/or the figures relate to yawning or weariness.

In some embodiments the video comprises at least one video of humans yawning and/or animals yawning.

In some embodiments the figure comprises at least one figure of humans yawning and/or animals yawning.

Without wishing to be bound by any theory or mechanism, yawning entails deep inhalation, which may improve drug delivery to the lungs, when using a nebulizer. Moreover, improvement of drug delivery to the lungs may lead to reduction of drug dosages, thus diminishing side effects. Yawning in humans is often triggered by sensing other yawning, and is a typical example of positive feedback. In other words, yawning may be contagious and subject to suggestibility.

In some embodiments the processing circuitry comprises a learning algorithm. In some embodiments the learning algorithm is configured to receive data relating to occurrences of said yawns in a subject and store the data in the non-transitory memory storage unit.

In some embodiments the data relating to occurrences of said yawns is derived from said still image, said dynamic image and/or said sound. In some embodiments the learning algorithm is configured to provide commands to the yawn stimulator based on said occurrences and said data, thereby enabling personalization of stimulation of yawning.

In some embodiments the yawning stimulation is activated manually. In some embodiments the yawning stimulation is manually activated by said subject or a caregiver.

In some embodiments the yawning stimulation is activated automatically. In some embodiments the yawning stimulation is activated automatically upon operation of the system. In some embodiments the yawning stimulation is activated automatically upon contact with the system. In some embodiments the yawning stimulation is activated automatically upon contact with the aerosol delivery device.

In some embodiments the aerosol delivery device is an inhaler or a nebulizer. In some embodiments the aerosol delivery device is an inhaler. In some embodiments the aerosol delivery device is a nebulizer.

In to some embodiments the aerosol delivery device comprises a container, configured to contain a liquid to be nebulized into said aerosols.

In some embodiments the aerosol delivery device comprises a nebulization chamber where the aerosols are produced.

In some embodiments the aerosol delivery device comprises a source of energy which creates the aerosol.

In some embodiments the source of energy comprises pneumatic energy or piezo-electric. In some embodiments the source of energy comprises pneumatic energy.

In to some embodiments the liquid comprises a pharmaceutical composition. In to some embodiments the aerosols comprise a pharmaceutical composition.

In some embodiments the pharmaceutical composition is for treating a pulmonary disease or disorder.

In some embodiments the pharmaceutical composition is selected from the group consisting of formoterol, albuterol, metaproterenol, terbutaline, bambuterol, clenbuterol, salmeterol, carmoterol, milveterol, indacaterol, saligenin- or indole-containing and adamantyl-derived β2 agonists, and pharmaceutically acceptable salts, esters, or isomers thereof. Each possibility represents a separate embodiment.

In some embodiments the pulmonary disease or disorder is selected the group consisting of asthma, inflammation, allergies, pulmonary vasoconstriction, allergic rhinitis, sinusitis, emphysema, impeded respiration, chronic obstructive pulmonary disease (COPD), pulmonary hypertension, bronchiectasis, respiratory distress syndrome parenchymatic and fibrotic lung diseases or disorders; cystic fibrosis, interstitial pulmonary fibrosis and sarcoidosis, tuberculosis and lung diseases and disorders secondary to HIV, pulmonary inflammation experienced with cystic fibrosis, and pulmonary obstruction experienced with cystic fibrosis. Each possibility represents a separate embodiment.

In some embodiments the aerosol delivery device is selected from the group consisting of: a pressurized meter dose inhaler, dry particle inhaler, soft mist inhaler, vibrating mesh nebulizer, jet nebulizer or ultrasonic wave nebulizer. Each possibility represents a separate embodiment of the present invention.

In some embodiments the aerosol delivery device is selected from the group consisting of: a pressurized meter dose inhaler, dry particle inhaler or soft mist inhaler. Each possibility represents a separate embodiment.

In some embodiments the aerosol release is a bolus aerosol release.

In some embodiments there is provided a use of a system as described herein in the treatment of a pulmonary disease or disorder.

In some embodiments the aerosols comprise a pharmaceutical composition for the treatment of said pulmonary disease or disorder.

In some embodiments the pharmaceutical composition is selected from the group consisting of formoterol, albuterol, metaproterenol, terbutaline, bambuterol, clenbuterol, salmeterol, carmoterol, milveterol, indacaterol, saligenin- or indole-containing and adamantyl-derived β2 agonists, and pharmaceutically acceptable salts, esters, or isomers thereof. Each possibility represents a separate embodiment.

In some embodiments there is provided a method of delivering aerosols to a subject in need thereof. The method comprising: providing an aerosol delivery device functionally associated with a processing circuitry having a yawn detector, wherein said aerosol delivery device comprises a controllable aerosol release mechanism; actuating the controllable aerosol release mechanism, upon the processing circuitry receiving indication of a yawn from the yawn detector, thereby releasing aerosols from the aerosol delivery device.

In some embodiments actuating the controllable aerosol release mechanism is performed automatically upon the processing circuitry receiving indication of a yawn from the yawn detector.

In some embodiments the method further comprises a step of receiving data relating to occurrences of said yawns in a subject by the processing circuitry. In some embodiments the method further comprises a step of storing the data in the non-transitory memory storage unit.

In some embodiments the method further comprises a step of gathering data relating to occurrences of said yawns in a subject.

In some embodiments gathering data comprises taking photos the user by the detector. In some embodiments gathering data comprises recording sounds of the user by the detector.

The terms “subject” and “user” as used herein are interchangeable. In some embodiments the subject is a human subject.

In some embodiments the method further comprises a step of stimulating yawning in the subject.

In some embodiments the stimulation of yawning is carried out by the processing circuitry. In some embodiments the stimulation of yawning is carried out by a yawn stimulation program in the processing circuitry.

In some embodiments the method is for the treatment of a respiratory disease or disorder.

In some embodiments treatment of a respiratory disease or disorder comprises alleviating shortness of breath.

In some embodiments the respiratory disease or disorder is selected from the group consisting of asthma, inflammation, allergies, pulmonary vasoconstriction, allergic rhinitis, sinusitis, emphysema, impeded respiration, chronic obstructive pulmonary disease (COPD), pulmonary hypertension, bronchiectasis, respiratory distress syndrome parenchymatic and fibrotic lung diseases or disorders; cystic fibrosis, interstitial pulmonary fibrosis and sarcoidosis, tuberculosis and lung diseases and disorders secondary to HIV, pulmonary inflammation experienced with cystic fibrosis, and pulmonary obstruction experienced with cystic fibrosis. Each possibility represents a separate embodiment.

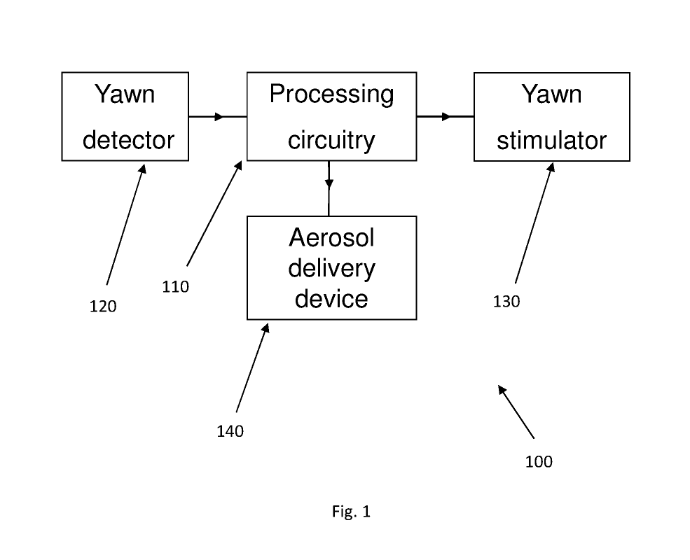

Reference is now made to FIG. 1, which schematically shows a functional block diagram of a system 100 for aerosols delivery, according to some embodiments. System 100 comprises a processing circuitry 110, which is functionally associated with a yawn detector 120, a yawn stimulator 130 and an aerosol delivery device 140.

Detector 120 is functionally associated with processing circuitry 110, meaning that it may have wireless or wired connection to processing circuitry 110. Yawn detector 120 is configured to receive and send electric signals to processing circuitry 110 through wired or wireless communication. Some of said electric signals are being referred to herein as yawn indicative signals.

In some embodiments yawn detector 120 is configured to detect motion, sound, pneumatic flow, change in pneumatic pressure, pneumatic pressure or any combination thereof. Appropriately, suitable detector may include video camera, still camera, microphone, EEG, microphone, motion detector, sound detector, air flow detector, air pressure detector and the like.

In some embodiments, system 100 may include a plurality of detectors, each functionally associated with processing circuitry 110, wherein each one of said detectors is configured to detect a different physical attribute. For example, system 100 may include a motion detector, such as a camera, and a pneumatic flow detector, located on a mouthpiece of aerosol delivery device 140, wherein both detectors are configured to receive and send electric signals to processing circuitry 110 through wired or wireless communication.

In some embodiments processing circuitry 110 comprises a computation unit, and may be an integral part of an external computer, such as a computation unit of a PC, laptop, smartphone, tablet and the like. In this case, yawn detector 120 may be an integral part of the mobile device, such as, but not limited to a camera and microphone of a smartphone.

In some embodiments processing circuitry 110 comprises a computation unit which is specifically designed for employment as a part of system 100.

In case that processing circuitry 110 is an integral part of an external computer, such as a computation unit of a mobile device, the detector may also be an integral part of the mobile device, such a camera and microphone of a smartphone.

Processing circuitry 110 is functionally associated with yawn detector 120, and is configured to receive a yawn indicative signal from it. Processing circuitry 110 is further configured to identify a yawn based on said yawn indicative signal. In some embodiments the identification of the yawn is facilitated by a recognition program installed in processing circuitry 110. The program may be, for example, as a part of the hardware of processing circuitry 110 or added thereto as a software or application.

Processing circuitry 110 is further configured to predict a yawn based on said yawn indicative signal. In some embodiments the prediction of the yawn is facilitated by the recognition program.

The recognition program installed in processing circuitry 110 may include for example a facial recognition program, an audio recognition program, air pressure recognition program, air flow recognition program and the like. For example, the recognition program installed in processing circuitry 110 may include algorithms for analyzing one or more of a facial gesture signals, audio signals, air pressure signals and/or air flow signals. In some embodiments, said algorithms are configured for providing output relating to predicting and/or determining when and/or if a yawn occurs, based on said analyzing. Appropriately, the yawn indicative signal may include any one or more of said facial gesture signals, audio signals, air pressure signals and/or air flow signals, such that the signals sent by yawn detector 120 to processing circuitry 110, are subsequently being analyzed by the recognition program and influence said output of the algorithm. The recognition program may be further configured to send a command to processing circuitry 110 indicating it to send or schedule a control signal to a controllable aerosol release mechanism of aerosol delivery device 140. The command may be based on said output of the algorithm.

In some embodiments, processing circuitry 110 is further configured predict a yawn based on the yawn indicative signal, and to provide a control signal to the aerosol release mechanism of aerosol delivery device 140, based on said prediction, thereby scheduling release of aerosols from aerosol delivery device 140. Similarly, processing circuitry 110 may be further configured to predict a yawn based said output of the algorithm, and to provide a control signal to the aerosol release mechanism of aerosol delivery device 140, based on said prediction, thereby scheduling release of aerosols from aerosol delivery device 140.

Processing circuitry 110 is also functionally associated with aerosol delivery device 140, meaning that it may have wireless or wired connection to delivery device 140. In particular, processing circuitry 110 may be functionally associated with a controllable aerosol release mechanism in aerosol delivery device 140, and configured to send it control signal, thereby affecting release of aerosols from delivery device 140. Processing circuitry 110 is further configured to schedule an aerosol release from aerosol delivery device 140 in a similar fashion.

In some embodiments processing circuitry 110 is equipped to receive the data specific to a user, comprising photos, videos and sounds of a user, provided by the detector. The processing circuitry 110 may incorporate this data in the recognition program, thereby modifying the algorithms for personalized yawn detection.

Processing circuitry 110 is further functionally associated with yawn stimulator 130, meaning that they may include wireless or wired connection. Optionally, both yawn stimulator 130 and processing circuitry 110 are incorporated into a single device, such as, but not limited to, a mobile device. In another option, yawn stimulator 130 and processing circuitry 110 are incorporated into a single device specifically designed for employment as a part of system 100.

Processing circuitry 110 provides data and commands to yawn stimulator 130 as to the stimulation of yawning. For example, processing circuitry 110 may include a non-transitory memory unit, where computer readable data is stored. Said computer readable data may correspond to yawn inducing elements, such as images, videos and sounds. Alternatively, said computer readable data may be stored in an external server, which is associated with processing circuitry 110 via wireless communication.

Yawn stimulator 130 is functionally associated with processing circuitry 110, and is configured to stimulate yawning in the user. Yawn stimulator 130 comprises a display element, such as a screen, and an audio element, such as speakers, which are configured to provide still images, dynamic images and sounds. Yawn stimulator 130 may receive commands from processing circuitry 110, relating to the stimulation of yawning in the user.

The sounds may include sound of humans yawning, sounds of animals yawing, pronunciations and/or repetitions of words and/or sentences, monotonic sounds and/or stories. Said words and/or sentences may be related to sleepiness and/or yawning, for example, the word ‘yawn’ and sentences indicating boredom weariness and desire to yawn. Said words and/or sentences and stories may be verbalized at a low pace thus further induce yawning.

The still images and dynamic images may include, but may not be limited to, video(s) and/or figure(s) of humans yawning, video(s) and/or figure(s) of animals yawing as well as video(s) and/or figure(s) related to yawning or weariness.

Without wishing to be bound by any theory or mechanism, yawning entails deep inhalation, which may improve drug delivery to the lungs, when using a nebulizer. Moreover, improvement of drug delivery to the lungs may lead to reduction of drug dosages, thus diminishing possible related side effects. Yawning in humans is often triggered by sensing other yawning, and is a typical example of positive feedback. In other words, yawning may be contagious and subject to suggestibility.

Optionally, both yawn stimulator 130 and processing circuitry 110 are incorporated into a single device, such as, but not limited to, a mobile device. In this case, the display element may be a screen and the audio element may be a speaker(s), both incorporated into the mobile device, for example, the screen and speakers of a smartphone. In another option, yawn stimulator 130 and processing circuitry 110 are incorporated into a single device specifically designed for employment as a part of system 100.

In some embodiments processing circuitry 110 further comprises a non-transitory memory storage unit and a learning algorithm. In this case, processing circuitry 110 is configured to receive data from yawn detector 120 relating to occurrences of yawns in a subject, and store the data in the non-transitory memory storage unit. Thereafter, the data is analyzed by the learning algorithm, and the learning algorithm may adjust the data and commands relating to yawn stimulation given by processing circuitry 110 to yawn stimulator 130, thereby enabling personalization of yawn induction.

Aerosol delivery device 140 comprises a pharmaceutical composition, a mouthpiece and a controllable aerosol release mechanism. The controllable aerosol release mechanism is configured to receive control signals from processing circuitry 110, thereby affecting the release of aerosols from aerosol delivery device 140. Aerosol delivery device 140 further comprises a source of energy which creates the aerosol. In some embodiments the source of energy comprises pneumatic energy or piezo-electric energy. Said source of energy is mechanically activated by the controllable aerosol release mechanism, upon receiving the control signal from processing circuitry 110. Immediately after forming, the aerosols are released through the mouthpiece.

The pharmaceutical composition is typically in the form of a solution, dispersion or suspension and is stored in a container, which may be refilled and/or replaced. The pharmaceutical composition is released as part of the aerosol such that the release of aerosols from aerosol delivery device 140 entails release of the pharmaceutical composition. The pharmaceutical composition comprises a pharmaceutically active ingredients for the treatment of pulmonary disease or disorder, and its release is generally intended to provide a therapeutic effect over said disease or disorder, or their symptoms.

In the case when yawn detector 130 comprises an air flow detector or a pressure detector, it is preferable that these are placed over the mouthpiece of aerosol delivery device 140, such that an accurate detection is made.

In some embodiments any one or more of yawn detector 130, processing circuitry 110 and yawn detector 120 may be placed over, or integrated with, aerosol delivery device 140, such that system 100 comprises a unified device.

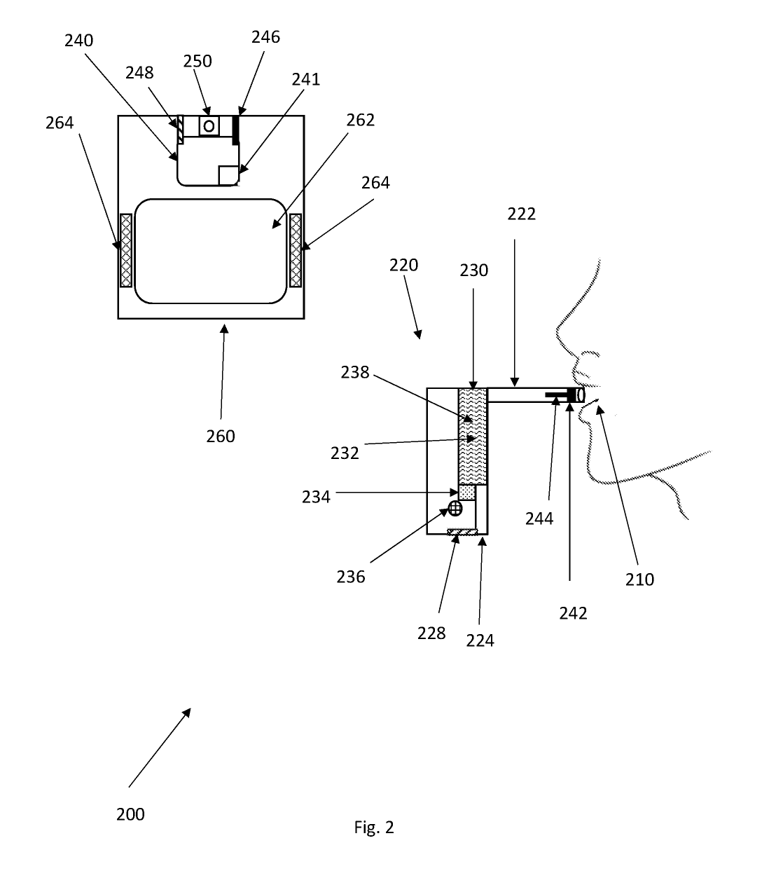

Reference is now made to FIG. 2, which schematically illustrates a system for aerosol delivery 200 comprising a nebulizer 220, wirelessly connected to a computation unit 240, which is functionally associated with an air flow meter 242, camera 250 and with a yawn stimulator 260 comprising a screen 262 and speakers 264.

Camera 250 has wired connection to computation unit 240. Camera 250 is configured to receive and send yawn indicative electric signals to computation unit 240. Camera 250 is configured to detect motion and to acquire electronic motion pictures. Camera 250 is further configured to transform said electronic motion pictures to computer readable data and to send said data to computation unit 240.

Air flow meter 242 is located at a mouthpiece 222 of nebulizer 220. Air flow meter 242 comprises a transmitter 244, which is configured to send electric signals to computation unit 240 through an antenna 246 of computation unit 240. Air flow meter 242 is wirelessly connected to computation unit 240 and is configured to wirelessly send yawn indicative electric signals to computation unit 240 using transmitter 244. Air flow meter 242 is configured to measure air flow and changes in air flow. Air flow meter 242 is further configured to transform said measurements to computer readable data and to send said data to computation unit 240.

Transmitter 244 is located on air flow meter 242 and is configured to translate measurements relating to air flow to electric signals transferrable by wireless communication.

Computation unit 240 is specifically designed for employment as a part of system 200. Computation unit 240 comprises antenna 246 and a transmitter 248.

Computation unit 240 is functionally associated with camera 250 and with air flow meter 242, and is configured to receive electronic signals from them. The electric signals of camera 250 are received through wired connection, whereas the electric signals of air flow meter 242 are received wirelessly through antenna 246.

Said electronic signals comprise computer readable data relating to electronic motion pictures and computer readable data relating to measure air flow and changes in air flow. Computation unit 240 is further configured to identify a yawn based on said computer readable data.

Computation unit 240 comprises a recognition program installed therein, such that said identification of a yawn is facilitated by said program based on said computer readable data. The recognition program installed in computation unit 240 includes algorithms for analyzing facial gestures associated with yawning, air flow values associated with yawning and changes thereof, which are indicative for yawning. Said algorithm is configured for providing output relating to predicting and/or determining when and/or if a yawn occurs, based on said analyzing. The recognition program is further configured to send a command to computation unit 240 which is in turn indicating it to send or schedule a control signal to a controllable aerosol release mechanism 224 of nebulizer 220. The command may be based on said output of the algorithm.

Computation unit 240 is further configured to predict a yawn based on the command, and to provide a control signal to the aerosol release mechanism 224 of nebulizer 220, based on said prediction, thereby scheduling release of aerosols from nebulizer 220.

Computation unit 240 comprises transmitter 248. Transmitter 248 is located on computation unit 240 and is configured to send wireless control signals to controllable aerosol release mechanism 224 of nebulizer 220, through an antenna 228 of controllable aerosol release mechanism 224. Consequently, computation unit 240 is functionally associated with controllable aerosol release mechanism 224 of nebulizer 220, and is configured to send thereto control signal(s), thereby affecting release of aerosols from nebulizer 220. Similarly, computation unit 240 is further configured to schedule an aerosol release from nebulizer 220 using said wireless control signals from transmitter 248 to antenna 228.

Computation unit 240 includes a non-transitory memory unit 241, where computer readable data is stored. The computer readable data includes data corresponding to yawn inducing elements, such as images, videos and sounds that induce yawning.

Computation unit 240 is connected to yawn stimulator 260. As can be seen in FIG. 2, yawn stimulator 260 and computation unit 240 are incorporated into a single device specifically designed for employment as a part of system 200. Computation unit 240 provides data and commands to yawn stimulator 260 as to the stimulation of yawn.

Antenna 246 is located on computation unit 240 and is configured to receive electronic signals from air flow meter 242 via transmitter 244. It is further configured to translate said electric signals transferrable by wireless communication to computer readable data and to transfer said data to computation unit 240.

Transmitter 248 is located on computation unit 240 and is configured to wirelessly transmit control signals to a controllable aerosol release mechanism 224 of nebulizer 220.

Yawn stimulator 260 comprises screen 262 and speakers 264. It is functionally associated with computation unit 240, and is configured to stimulate yawning in the user. Screen 262 is configured to provide still images and dynamic images. Speakers 264 are configured to provide sounds. Yawn stimulator 260 may receive commands from computation unit 240, relating to the stimulation of yawning in the user.

The sounds may include sound of humans yawning, sounds of animals yawing, pronunciations and/or repetitions of words and/or sentences, monotonic sounds and/or stories. Said words and/or sentences may be related to sleepiness and/or yawning, for example, the word ‘yawn’ and sentences indicating boredom weariness and desire to yawn. Said words and/or sentences and stories may be verbalized at a low pace thus further induce yawning.

The still images and dynamic images may include but not limited to, video(s) and/or figure(s) humans yawning, video(s) and/or figure(s) of animals yawing as well as video(s) and/or figure(s) related to yawning or weariness.

Nebulizer 220 comprises a container 230 comprising a liquid 232, a mouthpiece 222 and a controllable aerosol release mechanism 224. Nebulizer 220 is configured to release aerosols upon receiving a control signal from computation unit 240 to controllable aerosol release mechanism 224.

Controllable aerosol release mechanism 224 is configured to receive control signals from computation unit 240, thereby affecting the release of aerosols from nebulizer 220. Nebulizer 220 further comprises a pneumatic energy source 234, which creates the aerosol. Said control signal are received by antenna 228.

Antenna 228 is located on nebulizer 220 and is configured to receive electronic control signals from computation unit 240 via transmitter 248.

Pneumatic energy source 234 is located on container 230 and is configured to exert pneumatic energy on liquid 232, thereby nebulizing it and creating an aerosol. It is mechanically activated by controllable aerosol release mechanism 224, upon receiving the control signal from computation unit 240. Immediately after forming, the aerosols are released through mouthpiece 222.

Mouthpiece 222 is located at one end of nebulizer 220 and is designed to fit into a mouth of a user 210. Mouthpiece 222 is located in proximity to container 230, such that when used, the nebulized aerosols are ejected through mouthpiece 222 into the mouth of the user 210.

Container 230 is located inside nebulizer 220 and in close proximity to pneumatic energy source 234, to mouthpiece 222 and to manual switch 236. It comprises liquid 232, which may be refilled or otherwise, entire container 230, can be switched with a new, analogous container.

Liquid 232 is located inside container 230 and comprises a solution, dispersion or suspension comprising a pharmaceutical composition 238. Pharmaceutical composition 238 is released as part of the aerosol, such that the release of aerosols from nebulizer 220 entails release of pharmaceutical composition 238. Pharmaceutical composition 238 comprises a medicine for treatment of pulmonary disease or disorder, and its release is generally intended to induce a therapeutic effect over said disease or disorder, or over their symptoms.

Nebulizer 220 further comprises a manual switch 236, located on the controllable aerosol release mechanism 224, such that upon decision of a user, the user may press the switch, thereby actuate the pneumatic energy source 234 and affect a release of aerosols from container 230 into the user's mouth 210 through mouthpiece 222.

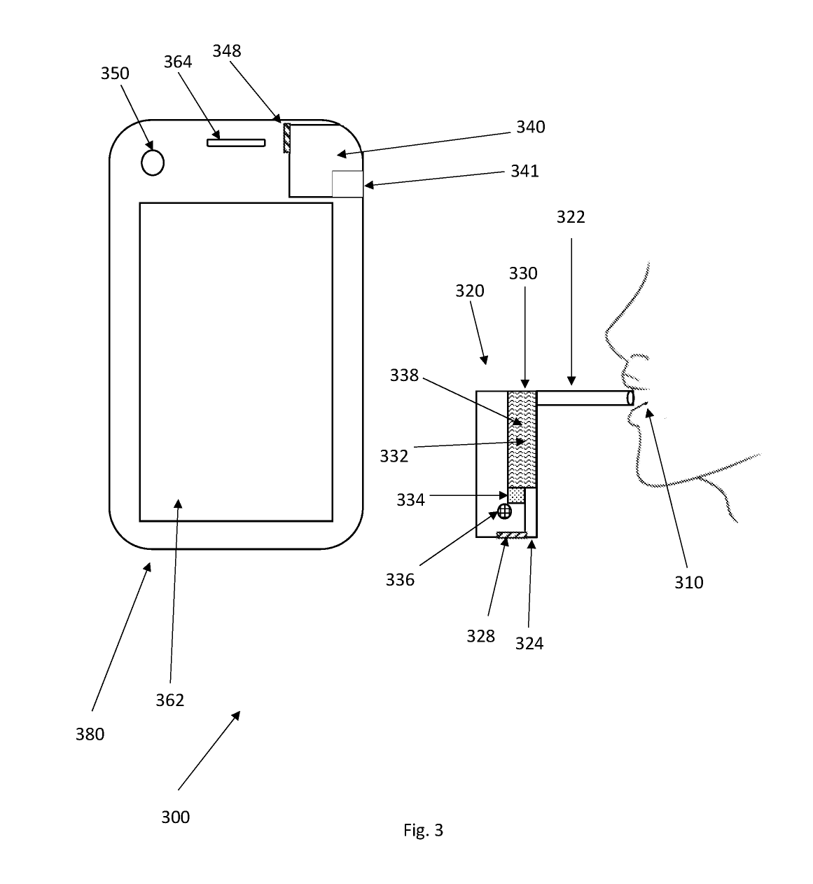

Reference is now made to FIG. 3, which schematically illustrates a system for aerosol delivery 300 comprising a nebulizer 320, wirelessly connected to a smartphone 380, comprising a computation unit 340, a camera 350, a screen 362, a speaker 364 and a transmitter 348.

Smartphone 380 may be any type of commercial smartphone, rather than specifically designed for system 300, as long as it includes a screen, speakers, camera and wireless communication through a transmitter, and as long as it includes an application or program as discussed hereinbelow.

Camera 350 is an integral part of smartphone 380. Camera 350 has wired connection computation unit 340. Camera 350 is configured to receive and send yawn indicative electric signals to computation unit 340. Camera 350 is configured to detect motion and to acquire electronic motion pictures. Camera 350 is further configured to transform, through a wired connection, said electronic motion pictures to computer readable data and to send said data to computation unit 340.

Computation unit 340 is an integral part of smartphone 380. It is associated with transmitter 348 via an electric connection. Computation unit 340 is functionally associated with camera 350 and is configured to receive electronic signals from it through wired connection.

Said electronic signals comprise computer readable data relating to electronic motion pictures. Computation unit 340 is further configured to identify a yawn based on said computer readable data.

Computation unit 340 comprises a recognition program installed therein, such that said identification of a yawn is facilitated by said program based on said computer readable data. The recognition program comprises a smartphone application or a program. The recognition program installed in computation unit 340 includes algorithms for analyzing facial gestures associated with yawning. Said algorithm is configured for providing output relating to predicting and/or determining when and/or if a yawn is expected tooccur, based on said analyzing. The recognition program is further configured to send a command to computation unit 340 indicating it to send or schedule a control signal to a controllable aerosol release mechanism 324 of nebulizer 320. The command is typically based on said output of the algorithm.

Computation unit 340 is further configured to predict a yawn based on the command, and to provide a control signal to the aerosol release mechanism 324 of nebulizer 320, based on said prediction, thereby scheduling release of aerosols from nebulizer 320.

Computation unit 340 is associated with transmitter 348 via an electric connection. Transmitter 348 is an integral part of smartphone 380 and is configured to send wireless control signals to controllable aerosol release mechanism 334 of nebulizer 320, through an antenna 328 of controllable aerosol release mechanism 324. Consequently, computation unit 340 is functionally associated with controllable aerosol release mechanism 324 of nebulizer 320, and is configured to send it control signals, thereby affecting release of aerosols from nebulizer 320. Similarly, computation unit 340 is further configured to schedule an aerosol release from nebulizer 320 using said wireless control signals from Transmitter 348 to antenna 328.

Computation unit 340 includes a non-transitory memory unit 341, where computer readable data is stored. The computer readable data includes data corresponding to yawn inducing elements, such as images, videos and sounds that induce yawning.

Computation unit 340, screen 362 and speaker 364 are integral parts of smartphone 380 and so, computation unit 340 is connected to screen 362 and speaker 364 through wires. Computation unit 340 provides data and commands to screen 362 and speaker 364 as to the images and sound they produce.

Transmitter 348 is an integral part of smartphone 380 and is configured to wirelessly transmit control signals to a controllable aerosol release mechanism 324 of nebulizer 320.

Smartphone 380 comprises screen 362 and speaker 364, both of which are functionally associated with computation unit 340, and are configured to stimulate yawning in the user. Screen 362 is configured to provide still images and dynamic images. Speaker 364 are configured to provide sounds. Both speaker 364 and screen 362 receive commands from computation unit 340, relating to the stimulation of yawning in the user.

The sounds may include sound of humans yawning, sounds of animals yawing, pronunciations and/or repetitions of words and/or sentences, monotonic sounds and/or stories. Said words and/or sentences may be related to sleepiness and/or yawning, for example, the word ‘yawn’ and sentences indicating boredom weariness and desire to yawn. Said words and/or sentences and stories may be verbalized at a low pace thus further induce yawning.

The still images and dynamic images may include, but are not limited to, video(s) and/or figure(s) of humans yawning, video(s) and/or figure(s) of animals yawing as well as video(s) and/or figure(s) related to yawning or weariness.

Nebulizer 320 comprises a container 330 comprising a liquid 332, a mouthpiece 322 and a controllable aerosol release mechanism 324. Nebulizer 320 is configured to release aerosols upon receiving a control signal from computation unit 340 to controllable aerosol release mechanism 324.

Controllable aerosol release mechanism 324 is configured to receive control signals from computation unit 340, thereby affecting the release of aerosols from nebulizer 320. Nebulizer 320 further comprises a pneumatic energy source 334, which creates the aerosol. Said control signals are received by antenna 328.

Antenna 328 is located on nebulizer 320 and is configured to receive electronic control signals from computation unit 340 via transmitter 348.

Pneumatic energy source 334 is located on container 330 and is configured to exert pneumatic energy on liquid 332, thereby nebulizing it and create an aerosol. It is mechanically activated by controllable aerosol release mechanism 324, upon receiving the control signal from computation unit 340. Immediately after forming, the aerosols are released through mouthpiece 322.

Mouthpiece 322 is located at one end of nebulizer 320 and is designed to fit into a mouth of a user 310. Mouthpiece 322 is located in proximity to container 330, such that when used, the nebulized aerosols are ejected through it into the mouth of the user 310.

Container 330 is located inside nebulizer 320 and in close proximity to pneumatic energy source 334 to mouthpiece 322 and to a manual switch 336. It comprises liquid 332, which may be refilled or otherwise, entire container 330, can be switched with a new, analogous container.

Liquid 332 is located inside container 330 and comprises a solution, dispersion or suspension comprising a pharmaceutical composition 338. Pharmaceutical composition 338 is released as part of the aerosol, such that the release of aerosols from nebulizer 320 entails release of pharmaceutical composition 338. Pharmaceutical composition 338 comprises a medicine for treatment of pulmonary disease or disorder, and its release is generally intended to have a therapeutic effect over said disease or disorder, or over its symptoms.

Nebulizer 320 further comprises a manual switch 336, located on the controllable aerosol release mechanism 324, such that upon decision of a user, the user may press the switch, thereby actuate the pneumatic energy source 334 and affect a release of aerosols from container 330 into his mouth 310 through mouthpiece 322.

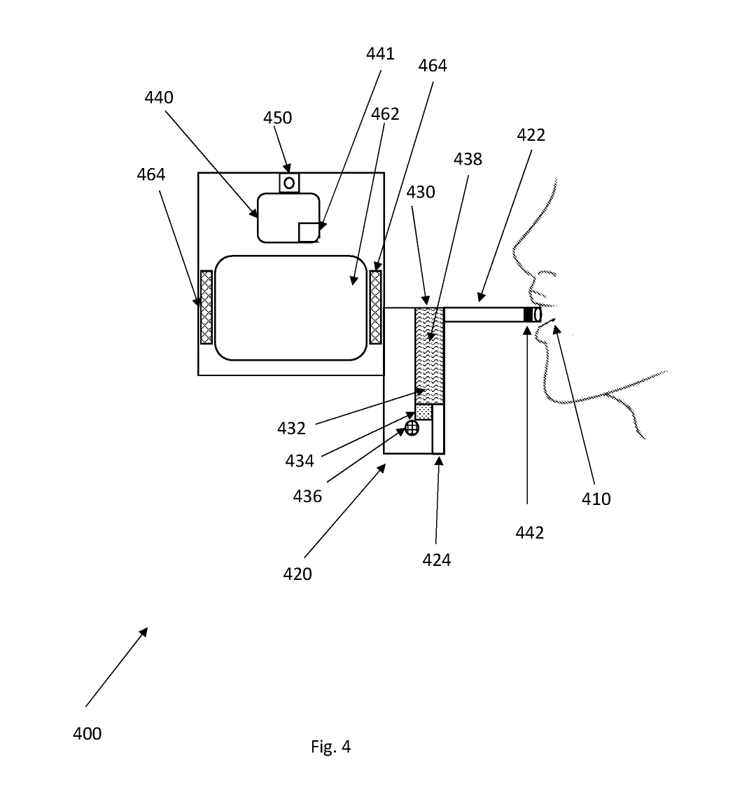

Reference is now made to FIG. 4, which schematically illustrates a device for aerosol delivery 400 comprising a nebulizer 420, a computation unit 440, a camera 450, an air flow meter 442, a screen 462, and speakers 464.

As can be seen in FIG. 4, each one of nebulizer 420, computation unit 440, camera 450, air flow meter 442, screen 462, and speakers 464 is an integral component of device 400, whereas each one of camera 450, air flow meter 442, screen 462, and speakers 464 is connected through wired connection to computation unit 440.

Camera 450 is an integral part of device 400. It has wired connection to computation unit 440. Camera 450 is configured to receive and send yawn indicative electric signals to computation unit 440. Camera 450 is configured to detect motion and to acquire electronic motion pictures. Camera 450 is further configured to wirely transform said electronic motion pictures to computer readable data and to send said data to computation unit 440.

Air flow meter 442 is located at a mouthpiece 422 of nebulizer 420. Air flow meter 442 is configured to send wired electric signals, such as yawn indicative electric signals, to computation unit 440. Air flow meter 442 is configured to measure air flow and changes in air flow. Air flow meter 442 is further configured to transform said measurements to computer readable data and to wirely send said data to computation unit 440.

Computation unit 440 is an integral part of device 400. It is functionally associated with camera 450 and is configured to receive electronic signals from it through wired connection. Computation unit 440 is functionally associated with camera 450 and with air flow meter 442, and is configured to receive wired electronic signals from them.

Said electronic signals comprise computer readable data relating to electronic motion pictures and computer readable data relating to measure air flow and changes in air flow. Computation unit 440 is further configured identify a yawn based on said computer readable data.

Computation unit 440 comprises a recognition program installed therein, such that said identification of a yawn is facilitated by said program based on said computer readable data. The recognition program installed in computation unit 440 includes algorithms for analyzing facial gestures associated with a yawn, air flow values associated with a yawn and changes thereof, which are indicative for a yawn. The recognition program may be a part of an added software or a part of a hardware component of computation unit 440.

The recognition program installed in computation unit 440 includes algorithms for analyzing facial gestures associated with a yawn. Said algorithm is configured for providing output relating to predicting and/or determining when and/or if a yawn occurs, based on said analyzing. The recognition program is further configured to send a command to computation unit 440 indicating it to send or schedule a control signal to a controllable aerosol release mechanism 424 of nebulizer 420. The command is typically based on said output of the algorithm.

Computation unit 440 is further configured to predict a yawn based on the command, and to provide a control signal to the aerosol release mechanism 424 of nebulizer 420, based on said prediction, thereby scheduling release of aerosols from nebulizer 420.

Computation unit 440 is wirely associated with controllable aerosol release mechanism 424 of nebulizer 420, and is configured to send it control signals, thereby affecting release of aerosols from nebulizer 420. Similarly, computation unit 440 is further configured to schedule an aerosol release from nebulizer 420.

Computation unit 440 includes a non-transitory memory unit 441, where computer readable data is stored. The computer readable data includes data corresponding to yawn inducing elements, such as images, videos and sounds that induce yawning.

Computation unit 440, screen 462 and speakers 464 are integral parts of device 400 and so, computation unit 440 is wirely connected to screen 462 and to speakers 464. Computation unit 440 provides data and commands to screen 462 and speakers 464 as to the images and sound they produce.

Both screen 462 and speakers 464 are functionally associated with computation unit 440, and are configured to stimulate yawning in the user. Screen 462 is configured to provide still images and dynamic images. Speakers 464 are configured to provide sounds. Both speakers 464 and screen 462 receive commands from computation unit 440, relating to the stimulation of yawning in the user.

The sounds may include sound of humans yawning, sounds of animals yawing, pronunciations and/or repetitions of words and/or sentences, monotonic sounds and/or stories. Said words and/or sentences may be related to sleepiness and/or yawning, for example, the word ‘yawn’ and sentences indicating boredom weariness and desire to yawn. Said words and/or sentences and stories may be verbalized at a low pace thus further induce yawning.

The still images and dynamic images may include but not limited to, video(s) and/or figure(s) of humans yawning, video(s) and/or figure(s) of animals yawing as well as video(s) and/or figure(s) related to yawning or weariness.

Nebulizer 420 comprises a container 430 comprising a liquid 432, mouthpiece 422 and controllable aerosol release mechanism 424. Nebulizer 420 is configured to release aerosols upon receiving a control signal from computation unit 440 to controllable aerosol release mechanism 424.

Controllable aerosol release mechanism 424 is configured to receive control signals from computation unit 440, thereby affecting the release of aerosols from nebulizer 420. Nebulizer 420 further comprises a pneumatic energy source 434, which creates the aerosol. Said control signals are received wirely from computation unit 440.

Pneumatic energy source 434 is located on container 430 and is configured to exert pneumatic energy on liquid 432, thereby nebulizing it and create an aerosol. It is mechanically activated by controllable aerosol release mechanism 424, upon receiving the control signal from computation unit 440. Immediately after forming, the aerosols are released through mouthpiece 422.

Mouthpiece 422 is located at one end of nebulizer 420 and is designed to fit into a mouth of a user 410. Mouthpiece 422 is located in proximity to container 430, such that when used, the nebulized aerosols are ejected through it into the mouth of the user 410.

Container 430 is located inside nebulizer 420 and in close proximity to pneumatic energy source 434, to mouthpiece 422 and to a manual switch 436. It comprises liquid 432, which may be refilled or otherwise, entire container 430, can be switched with a new, analogous container.

Liquid 432 is located inside container 430 and comprises a solution, dispersion or suspension comprising a pharmaceutical composition 438. Pharmaceutical composition 438 is released as part of the aerosol, such that the release of aerosols from nebulizer 420 entails release of pharmaceutical composition 438. Pharmaceutical composition 438 comprises a medicine for treatment of pulmonary disease or disorder, and its release is generally intended to have a mitigative effect over said disease or disorder, or over its symptoms.

Nebulizer 420 further comprises a manual switch 436, located on the controllable aerosol release mechanism 424, such that upon decision of a user, he may press the switch, thereby actuate the pneumatic energy source 434 and affect a release of aerosols from container 430 into his mouth 410 through mouthpiece 422.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

### EXAMPLES

### Example 1— Yawn-Induced Aerosol Inhalation

A user in need for an aerosol delivery operates the operating mode by pressing the on switch, located on the nebulizer and selects setup mode by pressing the setup switch, located next to the on switch. This turns on a device having a screen, speakers, a camera and an internal computer having a facial recognition program, all of which are integral with the nebulizer. The screen, camera and the built-in speakers are simultaneously operated by the internal computer. At first, the screen displays a live dynamic video of the user, being taken by the camera. Simultaneously, a massage appears on the screen indicating to the user that he is in setup mode and that he should insert the nebulizer into his mouth and avoid yawning. The massage further indicates that the user is about to be photographed by the camera in a non-yawning position. The massage further indicates the time in which the photograph is about to be taken using a countdown indication. When the countdown reaches zero, a photographs of the user is being taken and a computer readable data corresponding to the photograph is temporarily saved in a non-transitory memory within the computer.

The screen displays the photograph, received from the computer and a second massage asking the user weather he agrees that the photograph serves as a reliable indication of how he looks while not yawning. The user can affirm or refuse that it serves as a reliable indication of how he looks while not yawning. The user affirms by pressing a second switch, located next to the on switch, and so, the photo data is permanently stored and the computer readable data corresponding to the photograph is encoded in the facial recognition program. If the user would refuse (by pressing a third switch, located next to the second switch), the photo data would be erased. Weather the user affirms or refuses the indication, the process (i.e. of preparing and taking a photo) is repeated until ten indicative photographs of the user in a non-yawning position are stored. The user is repeating the process until ten indicative photographs in a non-yawning position are stored.

After data of the user in a non-yawning position is gathered, a second stage of the setup commences. A third massage appears on the screen indicating to the user that he is about to be presented with a video clip and sound. The third massage further indicates that immediately when the user starts yawning, he should press the second switch. The third massage further indicates that immediately when the user finishes yawning, he should press the second switch again. A video clip is displayed, and the camera starts documenting the user's facial feature and elements, which were programmed beforehand. The elements include, the user's mouth, lips eyes, jaw, ears, nose, nostrils, chin, cheeks, eyebrows, neck, facial skin and forehead. Throughout the documentation, computer readable data corresponding to the documentation is temporarily stored in the non-transitory memory within the computer.

After a minute the user starts yawning and immediately presses the second switch. After few seconds the user finishes yawning and presses the second switch again.

The screen displays a part of the video, corresponding to the period between the first and second presses of the second switch, as derived from the stored computer readable data in the computer.

A fourth massage asking the user weather he agrees that the video serves as a reliable indication of how he looks while yawning. The user can affirm or refuse that it serves as a reliable indication of how he looks while yawning. The user affirms by pressing the second switch, and so computer readable data corresponding to a first video and a computer readable data corresponding to a second video are stored in the non-transitory memory within the computer. The first video includes a part of the video, corresponding to the period between three seconds before the first press of the second switch and the first press of the second switch. The first video is indicative of pre-yawning facial gestures of the user. The second video includes a part of the video, corresponding to the period between the first press of the second switch and the second press of the second switch. The second video is indicative of yawning facial gestures of the user.

Both videos are stored and encoded in the facial recognition program, which includes a yawn detection algorithm and a pre-yawn detection algorithm.

If the user would refuse (by pressing the third switch), the computer readable video data would be deleted from the non-transitory memory within the computer. Weather the user affirms or refuses the indication, the process (i.e. of showing a clip and taking a video) is repeated until ten indicative videos of the user in a yawning position are stored. The user is repeating the process until ten indicative videos in a yawning position are stored. A fifth massage, indicating the completion of setup is presented on the screen. The facial recognition program constructs a personalized yawn detection algorithm for the user.

After a while, the user charges the nebulizer with a solution containing a pharmaceutical composition of salbutamol inside a fitted package.

The user inserts the nebulizer into his mouth operates the operating mode by pressing the on switch. This turns on the screen, speakers, camera and internal computer. As a result of the operation, the screen and speaker display and sound figures, videos, clips and sounds of people and animals yawing. Throughout the showing of the clip, the camera monitors the facial gestures of the user thus gathering visual data relating to his facial gestures. The visual data gathered from the camera is continuously translated the facial recognition program within the internal computer.

The user watches and listens to the clip and the show begins to induce yawning in the user. After about a minute, the user naturally shows pre-yawning facial gestures (i.e. facial signs indicative that a yawn is about to take place). The camera, which continuously monitors the facial gestures of the user, transfers the corresponding computer readable data to the facial recognition program within the computer, which analyzes the computer readable data, using the pre-yawn detection algorithm. The program recognizes that a yawn is about to occur and schedules command to eject a bolus of the pharmaceutical composition in about four seconds.

Throughout the next four seconds the camera still monitors facial gestures of the user and transfers the corresponding data to the program. The program perform further computations in order to re-evaluate the propensity towards a user's yawn in the forthcoming seconds. This feature enables a false positive control to avoid ejections of the pharmaceutical composition in a non-yawning condition. Moreover, based on the continuously gathered computer readable data, the program modifies the timing of the command to eject a bolus of the pharmaceutical composition.

After few seconds a yawn commences and a deep inhalation of the user occurs. A command is given by the computer to the nebulizer to eject a bolus containing the solution of the pharmaceutical composition. A bolus of the solution is ejected as a spray and inhaled by the user.

After a few seconds, a sixth massage appears on the screen asking the user if the ejection was properly timed during a deep inhalation. The user affirms by pressing the second switch and the algorithms are modified accordingly. The system, including the screen, speakers, camera and internal computer, shuts down.

1. A system for aerosols delivery, the system comprising:
an aerosol delivery device comprising a controllable aerosol release mechanism configured to release aerosols based on a control signal;
a yawn detector configured to provide a yawn indicative signal in a subject; and
a processing circuitry configured to identify a yawn based on said yawn indicative signal and to provide a control signal to said aerosol release mechanism, thereby affect release of aerosols from said device.

2. The system of claim 1, wherein identification of the yawn is facilitated by a facial recognition program capable of recognizing facial gestures associated with yawning.

3. The system of claim 1, wherein said processing circuitry is further configured to stimulate yawning in the subject.

4. The system of claim 3, further comprising a yawn stimulator configured to stimulate said yawning.

5. The system of claim 4, wherein said yawn stimulator is configured to provide a still image, dynamic image, sound, scent, flavor, sensation or any combination thereof.

6. The system of claim 3, wherein said yawning stimulation is activated by said subject or a caregiver.

7. (canceled)

8. (canceled)

9. The system of claim 1, wherein the aerosol delivery device is selected from the group consisting of: a pressurized meter dose inhaler, dry particle inhaler, soft mist inhaler and a nebulizer.

10. The system of claim 2, wherein said facial gestures comprise a deep inhalation maneuver.

11. The system of claim 1, wherein the processing circuitry is configured to predict a yawn based on the yawn indicative signal, and to provide a control signal to the aerosol release mechanism, based on said prediction, thereby schedule release of aerosols from the aerosol delivery device.

12. The system of claim 1, wherein said aerosol release is a bolus aerosol release.

13. The system of claim 1, wherein said aerosol comprises a pharmaceutical composition.

14. (canceled)

15. (canceled)

16. A method of delivering aerosols to a subject in need thereof, the method comprising
providing an aerosol delivery device functionally associated with a processing circuitry having a yawn detector, wherein said aerosol delivery device comprises a controllable aerosol release mechanism; and
actuating the controllable aerosol release mechanism, upon the processing circuitry receiving indication of a yawn from the yawn detector, thereby releasing aerosols from the aerosol delivery device.

17. The method of claim 16, further comprising stimulating a yawn in said subject.

18. The method of claim 16, wherein said receiving indication of a yawn comprises applying a facial recognition program capable of recognizing facial gestures associated with yawning.

19. The method of claim 17, wherein said stimulating a yawn comprises providing yawn stimulating signals.

20. The method of claim 19, wherein said yawn stimulating signals are selected from the group consisting of still image, dynamic image, sound, scent, flavor, sensation or a combination thereof.

21. (canceled)

22. The method of claim 16, wherein the aerosol delivery device is selected from a group consisting of: a pressurized meter dose inhaler, dry particle inhaler, a soft mist inhaler and a nebulizer.

23. (canceled)

24. The method of claim 16, wherein said aerosol release is a bolus aerosol release.

25. The method of claim 16, wherein said aerosol comprises a pharmaceutical composition.

26. The method of claim 16, for the treatment of a respiratory disease or disorder.

27. (canceled)
